(19) United States
(12) Patent Application Publication
(10) Pub. No.: US 2025/0143927 A1
(43) Pub. Date: May 8, 2025

(54) HEARING PROTECTION DEVICE WITH THERAPEUTIC COOLING MODULE

(71) Applicant: You Tune Hearing Protection LLC, Dayton, OH (US)

(72) Inventor: Jeremiah Smith, Dayton, OH (US)

(21) Appl. No.: 19/003,523

(22) Filed: Dec. 27, 2024

Related U.S. Application Data
(63) Continuation-in-part of application No. 18/341,288, filed on Jun. 26, 2023.
Application No. 18/341,288 is a continuation of application No. 17/986,541, filed on Nov. 14, 2022, now abandoned.
(60) Provisional application No. 63/279,934, filed on Nov. 16, 2021.

(51) Int. Cl. A61F 11/12; A61F 7/00; A61F 7/12

(52) U.S. Cl. CPC A61F 11/12; A61F 7/12; A61F 2007/0075

(57) ABSTRACT

Hearing protection devices are described that provide therapeutic benefit by a cooling mechanism adapted to lower the temperature of a user wearing the hearing protection device. The hearing protection devices include a cooling assembly; and an earpiece magnetically attachable to the cooling assembly, wherein the cooling assembly comprises a cooling module, a heat sink module in thermal communication with the cooling module, and a fan adapted to reject heat from the cooling module; the cooling module comprises a thermoelectric cooler and a cooler magnet; and the earpiece comprises a body, a thermally conductive insert having a thermally conductive tip portion, and an earpiece magnet configured to magnetically attract to the cooler magnet.

### CROSS-REFERENCES TO RELATED APPLICATIONS

This application is a continuation-in-part of U.S. patent application Ser. No. 18/341,288, filed Jun. 26, 2023, which is a continuation of U.S. patent application Ser. No. 17/986,541, filed Nov. 14, 2022, now abandoned, which claims benefit of priority under 35 U.S.C. § 119(e) to U.S. Provisional Application Ser. No. 63/279,934, filed Nov. 16, 2021. All of the aforementioned priority applications are hereby incorporated by reference herein in their entirety.

### TECHNICAL FIELD

The present disclosure relates generally to hearing protection devices and, more particularly, to a hearing protection device having a therapeutic cooling module that lowers the temperature of the inner ear, to cooling modules for the hearing protection device, and to therapeutic methods for using the hearing protection devices.

### BACKGROUND

Hearing loss significantly impacts individual well-being and the global economy, a fact well-documented in numerous studies. Despite substantial federal funding, no FDA-approved drug exists for hearing preservation or restoration. While promising research directions have been identified, these do not yet constitute a cure, and an FDA-approved treatment may still be a decade away.

Adult mammals lack the natural ability to regenerate cochlear sensory cells, as the cochlea does not possess the plasticity necessary for this regeneration. Research has explored various strategies, including rejuvenating cochlear hair cells, gene-based interventions, focused receptor blockers to prevent cochlear damage, and therapies aimed at enhancing hair cell regeneration following noise-related trauma. However, the complexity of regenerating sensory cells presents additional challenges, such as ensuring that regrown cells are tonotopically organized, innervated, capable of sending accurate neural signals, and free from abnormal growths. Drug delivery methods, such as local application through the eardrum into the middle ear, are not practical for field deployment or administration in impoverished countries.

Mild therapeutic hypothermia (MTH) involves controlled lowering of in-ear (cochlear) temperature by 1-5 degrees Celsius, offering an alternative strategy to counteract hearing loss due to acoustic trauma, ischemia-induced damage, and cisplatin therapy. MTH has been applied clinically in diverse fields, including brain and cardiac surgery. Cooling reduces inflammation and oxidative stress following numerous injuries while being safe and effective. MTH offers a quick intervention without needing pharmacological treatments or invasive procedures that could cause additional tissue damage. Therapeutic cooling protects cochlear hair cells and synapses from degeneration while attenuating the development of noise-induced hearing loss (NIHL).

It has been observed that by cooling the temperature of the human inner ear from normal body temperature of about 37° C. by about 1° C. to 3° C., mild therapeutic benefits may be realized. By cooling the temperature of the human to approximately 30° C., benefits for caloric vestibular testing may be realized. Particularly in high-noise environments, it has been observed that noise-related trauma may be slowed or even stopped if the temperature inside the ear is lowered. Devices that attempt to lower inner-ear temperatures by cooling external anatomy such as the mastoid bone surrounding the ears are not effective at lowering inner-ear temperature by a reasonable amount and within a reasonable time. Decreasing the temperature of the inner ear is further complicated in vivo, because blood flowing in the vicinity of the inner ear counteracts external stimuli, owing to the body's normal temperature regulation processes.

### SUMMARY

Some embodiments of the present disclosure are directed to hearing protection devices. The hearing protection devices include a cooling assembly an earpiece magnetically attachable to and detachable from the cooling assembly. The cooling assembly includes a cooling module, a heat sink module in thermal communication with the cooling module, and a fan adapted to reject heat from the cooling module. The cooling module includes a thermoelectric cooler and a cooler magnet configured to magnetically attach to the earpiece. The earpiece includes an earpiece body, a thermally conductive insert insertable into an ear canal and having a thermally conductive tip.

Some embodiments of the present disclosure are directed to cooling assemblies for the hearing protection devices. The cooling assemblies include a cooling module having a thermoelectric cooler and a cooler magnet adapted to magnetically attach the cooling module to a contact surface of a thermally conductive insert of an earpiece. The cooling assemblies also include a cooling module cover covering the cooling module, the cooling module cover having a thermal transfer surface configured to establish thermal communication with the contact surface. The cooling assemblies also include a heat sink module in thermal communication with the cooling module and a fan adapted to reject heat from the cooling module.

Some embodiments of the present disclosure are directed to headphone systems that include two of the cooling assemblies. Specifically, the two cooling assemblies include a first cooling assembly configured to be place over a left ear of a user and a second cooling assembly configured to be placed over a right ear of the user. The cooling assemblies are physically connected by a headband connecting the two hearing protection devices, so that the headphone system is wearable by the user.

Some embodiments of the present disclosure are directed to methods for mitigating hearing trauma to the ears of a user with the previously described headphone system. The methods include inserting the first earpiece into the left ear of the user; inserting the second earpiece into the right ear of the user; magnetically attaching the first cooling assembly to the first thermally conductive insert of the first earpiece to establish thermal communication between the first cooling module and the first conductive tip portion of the first earpiece; magnetically attaching the second cooling assembly to the second thermally conductive insert of the second earpiece to establish thermal communication between the second cooling module and the second conductive tip portion of the second earpiece; and activating the thermoelectric coolers of the cooling assemblies to decrease ambient temperature in the ear canal of the user.

Features and advantages of the embodiments described herein will be set forth in the detailed description that follows, and in part will be readily apparent to those skilled in the art from that description or recognized by practicing the embodiments described herein, including the detailed description that follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 is a schematic of the hearing protection device according to embodiments, including a cooling assembly and an earpiece.

FIG. 2 is a schematic of a headphone system including the hearing protection device according to embodiments.

FIG. 3 is a schematic of a fan-type cooling assembly according to embodiments.

FIG. 4 is a schematic of a blower-type cooling assembly according to embodiments.

FIGS. 5A and 5B are perspective views of the fan-type cooling assembly.

FIGS. 6A and 6B are transparent perspective views of the fan-type cooling assembly.

FIG. 7 is a side view of the fan-type cooling assembly.

FIG. 8 is a perspective view of the fan-type cooling assembly.

FIGS. 9A and 9B are perspective views of the blower-type cooling assembly.

FIGS. 10A and 10B are perspective views of the blower-type cooling assembly.

FIG. 11 is a cross-section of the blower-type cooling assembly.

FIG. 12 is a side view of the cooling module of the cooling assembly according to one embodiment.

FIG. 13 is a side view of the cooling apparatus of the cooling assembly according to one embodiment.

FIG. 14 is a front view of a screw for attaching the heat sink to the cooling assembly.

FIGS. 15A and 15B are perspective views of components of the earpiece according to embodiments.

FIG. 16 is a schematic of the earpiece fitted into the ear canal of a user.

FIGS. 17A, 17B, 17C, 17D, and 17E are multiple views of thermally conductive portions of earpieces.

FIGS. 18A and 18B are perspective views of components of a universal-fit earpiece.

FIGS. 19A, 19B, and 19C are perspective views of components of a universal-fit earpiece.

FIGS. 20A and 20B are views of an insert component of the universal-fit earpiece.

FIG. 21 is a front view of a thermally conductive tip component for the universal-fit earpiece.

### DETAILED DESCRIPTION

Embodiments of hearing protection devices described herein provide such temperature lowering of the ear in a convenient and portable manner that is less susceptible to being canceled by physiological temperature regulation from nearby blood flow.

Reference will now be made in detail to embodiments of hearing protection devices, cooling assemblies compatible with the hearing protection devices, and headphone systems including two hearing protection devices in tandem with two cooling assemblies. The headphone systems may be implemented into methods for mitigating hearing trauma to the ears of a user. The cooling assembly includes mechanisms adapted to remove heat from a contact point. The contact point of the cooling assembly is attachable, for example magnetically, to a thermally conductive portion of an earpiece that is inserted into the ear canal of a user. This approach offers an alternative strategy to counteract hearing loss caused by acoustic trauma, ischemia-induced damage, and cisplatin therapy. The magnetic connection facilitates ease of use, allowing the user to insert custom thermally conductive (e.g., aluminum or copper) ear molds into the ear canal and then magnetically attach the cooling assembly. A modular design addresses known limitations by enabling precise thermal modulation closer to the cochlea, improving cooling efficiency and comfort for extended use.

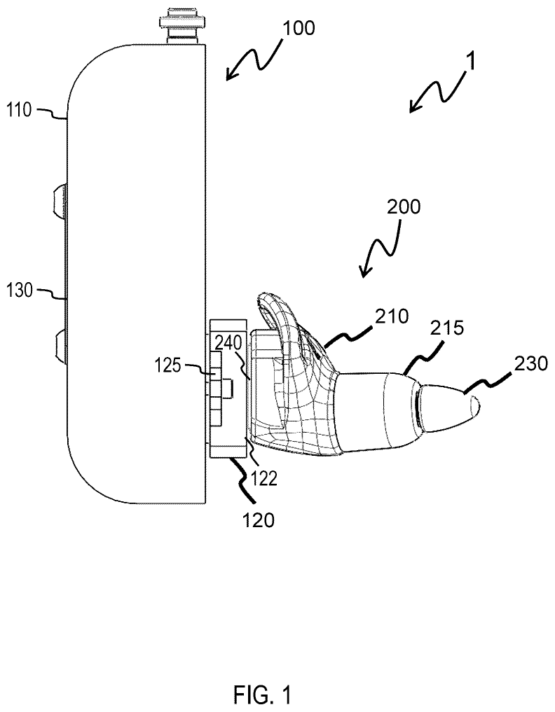

Referring to the example embodiment of FIG. 1, a hearing protection device 1 includes a cooling assembly 100 and an earpiece 200 magnetically attachable to the cooling assembly 100. In the hearing protection device 1 of FIG. 1, the cooling assembly 100 is illustrated as being magnetically attached to the earpiece 200. It should be understood that the cooling assembly 100 of FIG. 1 may be detached from earpiece 200 by pulling the cooling assembly 100 away from the earpiece 200.

In the hearing protection device 1, the cooling assembly 100 includes a cooling module 120, a heat sink module 110 in thermal communication with the cooling module 120, and a fan 130 adapted to reject heat from the cooling module 120. The cooling module 120 includes a thermoelectric cooler 125 and a cooler magnet 122.

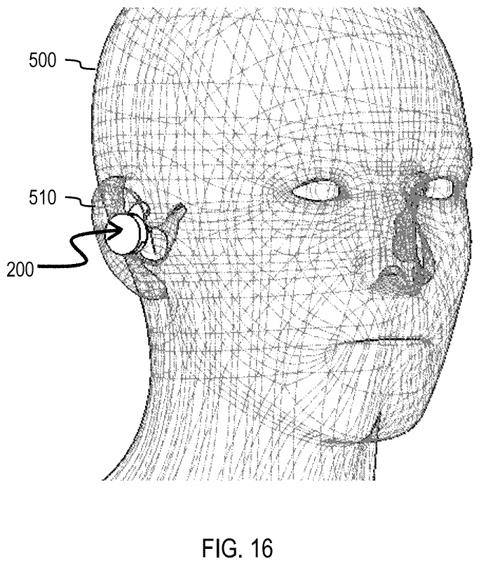

The earpiece 200 includes an earpiece body 210, a thermally conductive insert 220 (not visible in FIG. 1; see FIGS. 15A and 15B) inserted through the earpiece body 210. The earpiece 200 has a thermally conductive tip portion 230, and an earpiece magnet 240 configured to magnetically attract to the cooler magnet 122. When the earpiece 200 is inserted into the ear canal of a user, the thermally conductive tip portion 230 is located inside the ear canal, and the earpiece magnet 240 is outside the ear canal to enable connection of the earpiece magnet 240 to the cooler magnet 122 of the cooling assembly 100. Thereby, the hearing protection device 1 is configured so that heat is removed from the ear canal out the thermally conductive insert 220, into the cooling assembly 100, to be rejected to the external environment from the cooling assembly 100.

Specific embodiments herein are directed to cooling assemblies in isolation or in combination with the earpiece 200 as a hearing protection device 1.

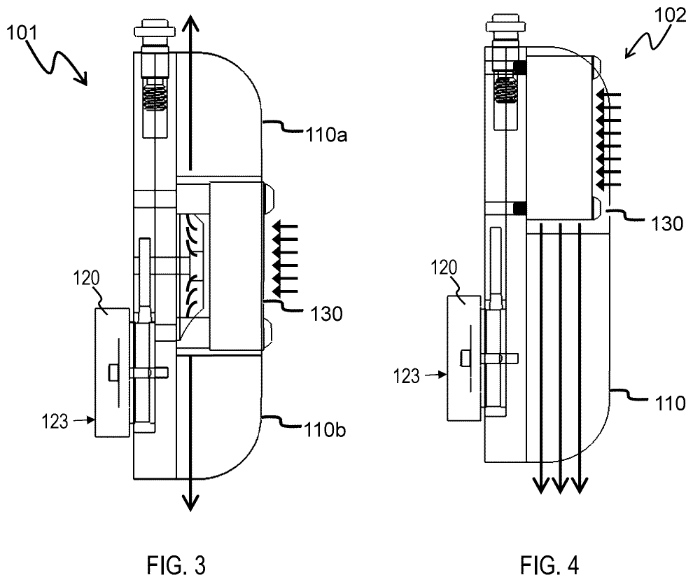

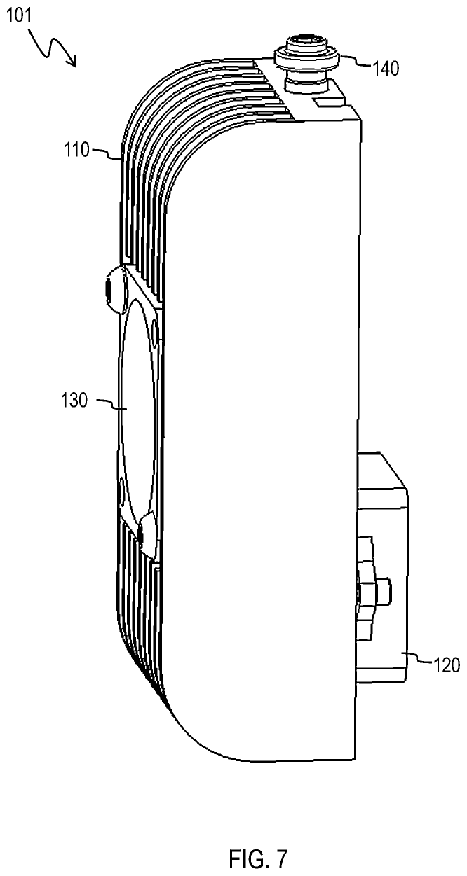

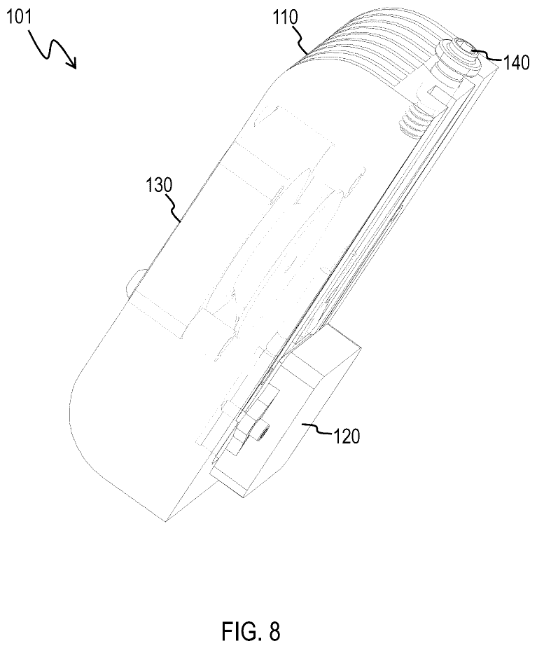

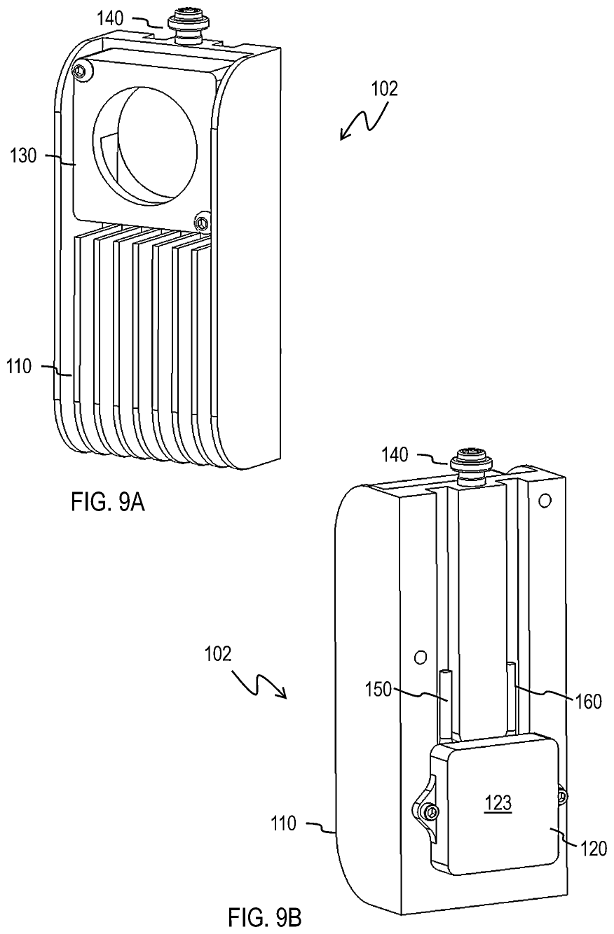

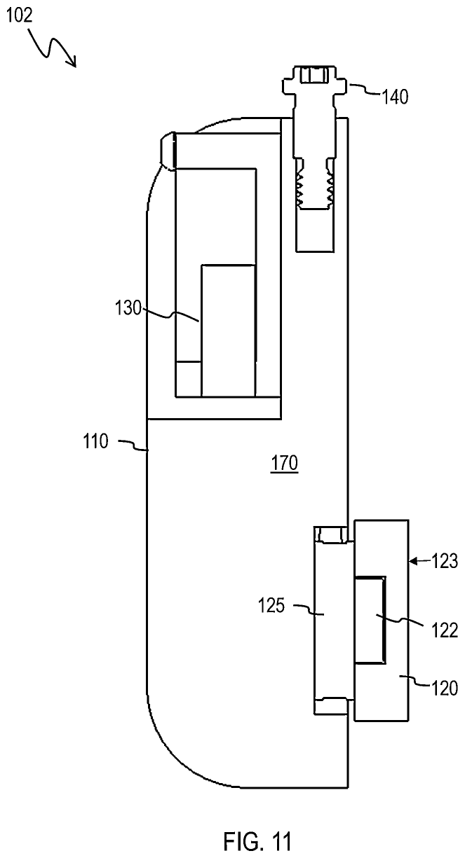

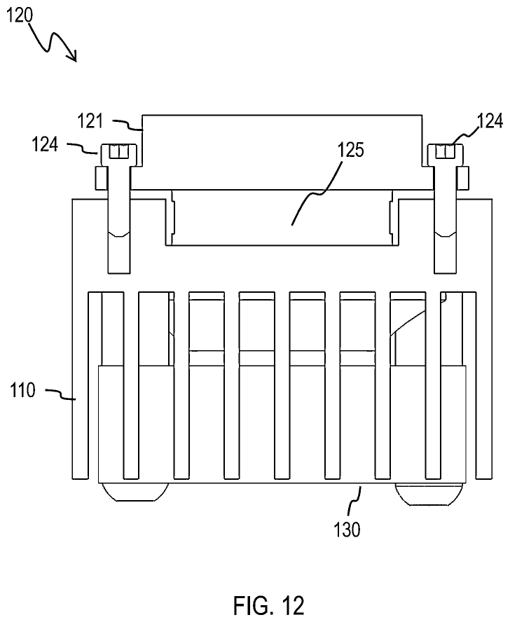

The cooling assemblies include a cooling module comprising a thermoelectric cooler and a cooler magnet; a heat sink module in thermal communication with the cooling module; and a fan adapted to reject heat from the cooling module. The components of the cooling assemblies will be described subsequently in detail, with reference to FIGS. 3-13.

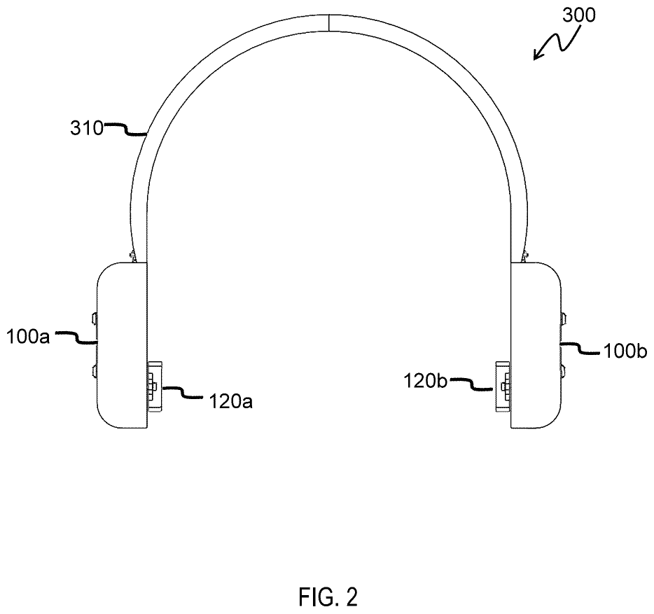

Referring to FIG. 2, embodiments herein are directed to a headphone system 300 including two cooling assemblies 100a, 100b configured to be placed over the ears of a user; and a headband 310 connecting the two hearing protection devices 100a, 100b. The headphone system 300 may further include a two hearing protection systems 1 (FIG. 1) including two earpieces 200, each magnetically attachable one of the two cooling assemblies 100a, 100b, each earpiece 200 including an earpiece body 210, a thermally conductive insert having a thermally conductive tip 230 portion for insertion into the ear of a user, and an earpiece magnet 240 configured to magnetically attract to the cooler magnet.

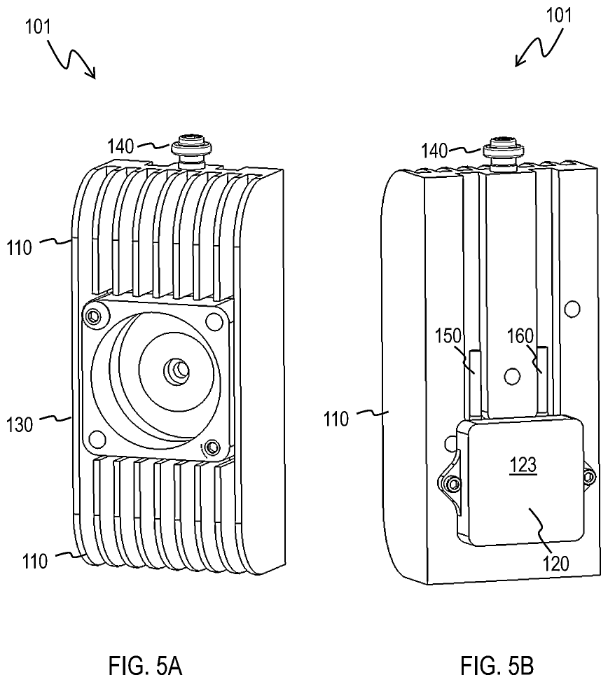

The cooling assembly 100 will now be described in detail, according to embodiments. FIGS. 3 and 4 are side views of two embodiments of the cooling assembly 100. Specifically, FIG. 3 is a side view of a fan-type cooling assembly 101, and FIG. 4 is a side view of a blower-type cooling assembly 102. Both the fan-type cooling assembly 101 and the blower-type cooling assembly 102 include a heat sink 110, 110a, 110b, a cooling module 120, and a fan 130. During operation of the hearing protection device 1, the cooling module removes heat from the earpiece and rejects heat away from the earpiece toward the cooling assembly. The configurations of FIGS. 3 and 4 are embodiments of cooling modules that handle the rejected heat slightly differently.

In the fan-type cooling assembly 101, the fan 130 is disposed between two heat sinks 110a, 110b. In this arrangement, cooling air is drawn inwardly to circulate the air through the body of the cooling assembly 100 as depicted by the arrows in FIG. 3, then cause heat to be rejected from the heat sinks 110a, 110b in two directions.

In the blower-type cooling assembly 102, the heat sink 110 is disposed opposite the cooling module 120, whereby heat from the cooling module 120 is transferred directly to the heat sink 110. The fan 130 is disposed above the heat sink 110 and aims cooling air directly across the fins of the heat sink 110 as depicted by arrows in FIG. 4.

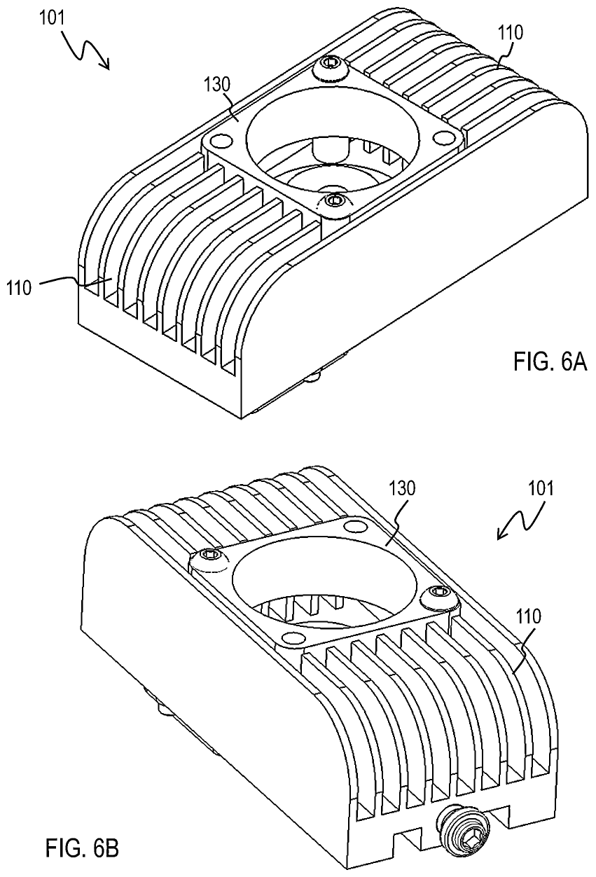

FIGS. 5A, 5B, 6A, 6B, 7, and 8 are various perspective views of the fan-type cooling assembly 101 as previously described. The fan-type cooling assembly 101 further includes a headphone connector 140 for connecting the fan-type cooling assembly 101 to a headband for use in a headphone system 300 as previously described (FIG. 2). The fan-type cooling assembly 101 further includes electrical connectors 150, 160 for connecting the cooling module 120 to a power source (not shown).

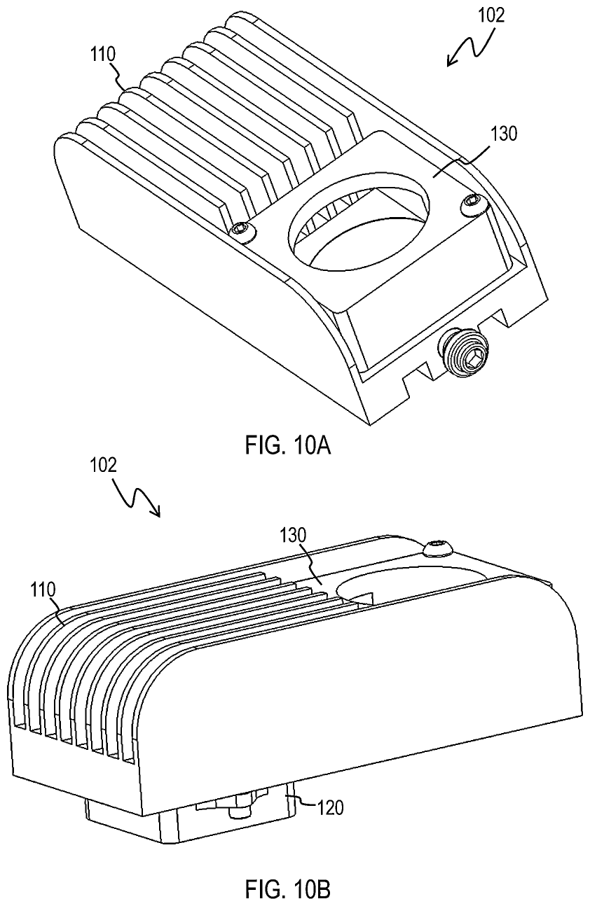

FIGS. 9A, 9B, 10A, 10B, and 11 are various perspective views of the blower-type cooling assembly 102 as previously described. The blower-type cooling assembly 102 further includes a headphone connector 140 for connecting the blower-type cooling assembly 102 to a headband for use in a headphone system 300 as previously described (FIG. 2). The blower-type cooling assembly 102 further includes electrical connectors 150, 160 for connecting the cooling module 120 to a power source (not shown).

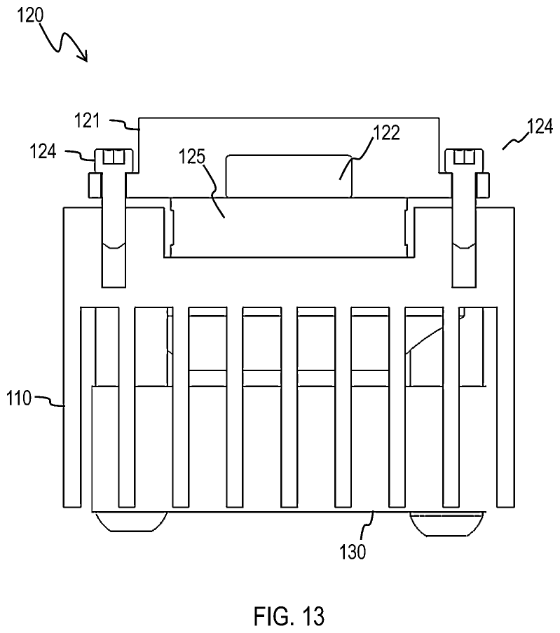

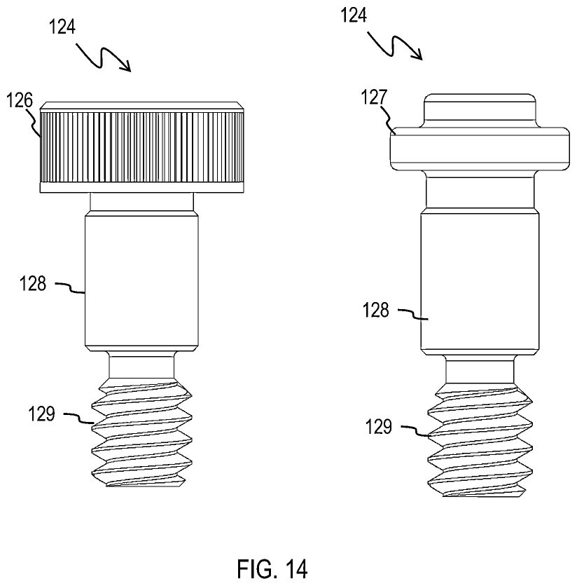

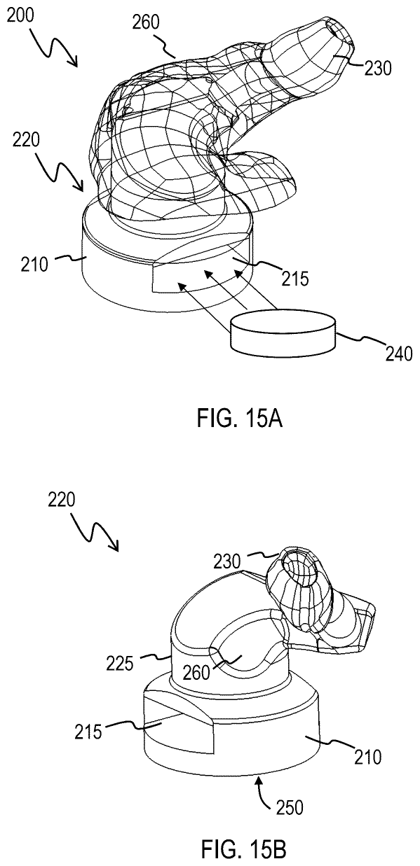

FIGS. 12 and 13 are cross sections of two embodiments of the cooling module 120 of the cooling assembly 100. The cooling module 120 includes a thermoelectric cooler 125 or Peltier device seated between the body of the cooling assembly and a cooling module cover 121 having a thermal transfer surface 123 to which the earpiece 200, specifically the earpiece magnet 240 and/or the thermally conductive insert 220 is magnetically attachable to the cooling module 120 at a contact surface 250 (see FIG. 15B) of the thermally conductive insert 220. The cooling module cover 121 is made of a material having suitable thermal conductivity to enable efficient thermal communication between the thermoelectric cooler 125 and the thermally conductive insert of the earpiece. The cooling module 120 may further include a cooling magnet 122 between the thermoelectric cooler 125 and the cooling module cover 121. The cooling magnet may be any type of magnet, such as a rare-earth magnet, for example. The cooling module 120 may further include cooling module screws 124. An example cooling module screw 124 is shown in FIG. 14 and includes a screw shaft 128 having screw threads 129. The cooling module screw 124 includes a screw head 127 that is covered by a screw cap 126 configured to facilitate the user's ability to rotate the cooling module screws 124 by 180 degrees and thereby adjust the angle of the cooling module cover 121 for an optimal fit flat against the earpiece.

As with any thermoelectric device, the thermoelectric cooler 125 of the cooling module 120 may be operated in a cooling mode or in a heating mode. Though for therapeutic benefits and injury mitigation the cooling mode is particularly important, for routine clinical vestibular testing, the ability to operate the thermoelectric cooler 125 also in a heating mode becomes beneficial. In vestibular testing, the inner ear of a patient is thermally cycled from normal body temperature (about 37° C.) through a reduced-temperature state (down to about 30° C.) back to a normal body temperature state and even to a slightly elevated temperature state. The ability to manipulate the temperature of the inner ear through such a cycle in an efficient and reproducible manner is therefore a particularly noteworthy benefit of the thermoelectric cooler in combination with a customizable thermally conductive insert.

Components of the earpiece 200 (FIG. 1) will now be described. Referring to the example embodiment of FIGS. 15A and 15B, the earpiece 200 includes a thermally conductive insert 220. The thermally conductive insert 220 includes an earpiece base 210, an earpiece body 225, and an earpiece tip 230. The earpiece 200 may further include an earpiece magnet slot 215 in the earpiece base 210 for accommodating an earpiece magnet 240 that is configured to attract the cooling magnet 122 in the cooling module 120. The earpiece 200 includes a contact surface 250 that is essentially flat to optimize thermal communication of the earpiece tip 230 with the cooling module 120. The earpiece 200 may further include a comfort covering 160 over the earpiece body 225. The comfort covering 160 may be a soft, molded material such as silicone, or may be a foam tip or other material that provides comfort to the user than the thermally conductive earpiece tip 230 is inserted into the ear of the user.

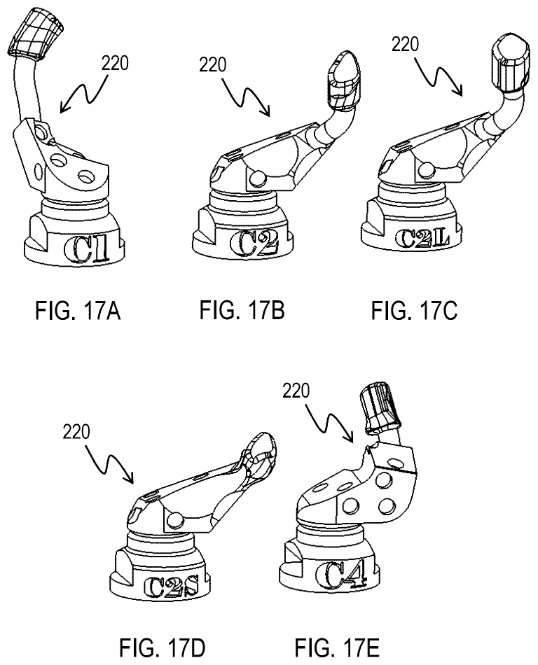

FIG. 16 schematically depicts the earpiece 200 of FIGS. 15A and 15B inserted into the ear 510 of a user 500.

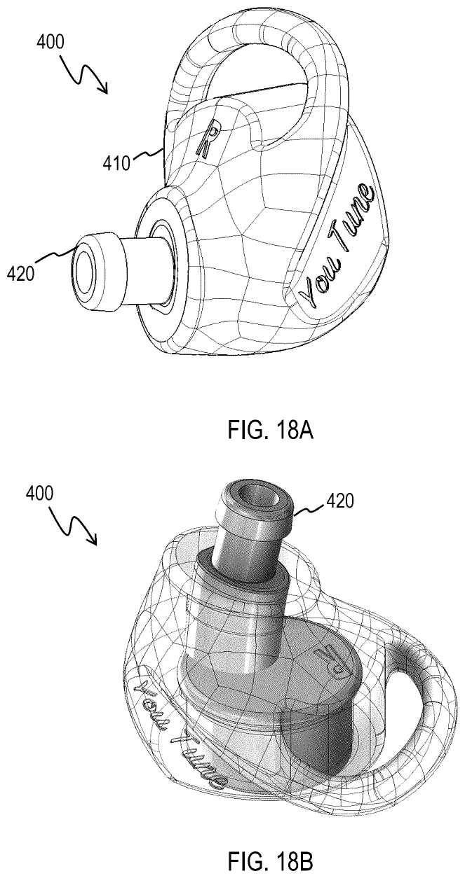

FIGS. 17A, 17B, 17C, 17D, and 17E are views of various configurations of thermally conductive inserts 220 for use within the earpiece. The thermally conductive inserts 220 are made from a thermally conductive material. Examples of thermally conductive materials include without limitation copper, aluminum, aluminum/copper alloys, and other alloys including copper and/or aluminum. The thermally conductive inserts 220 may be customized to the contours and dimensions of a patient or user's inner ear by first taking a mold of the inner ear by known techniques, then casting or 3D printing a thermally conductive insert with a thermally conductive material from the mold by known casting or 3D printing techniques. As should be evident from the various configurations in FIGS. 17A, 17B, 17C, 17D, and 17E, the thermally conductive insert 220 may vary in shape, angles, length, and thickness to enable positioning of the thermally conductive tip as close as physiologically practical to the eardrum of the patient or user, thereby enabling efficient temperature control.

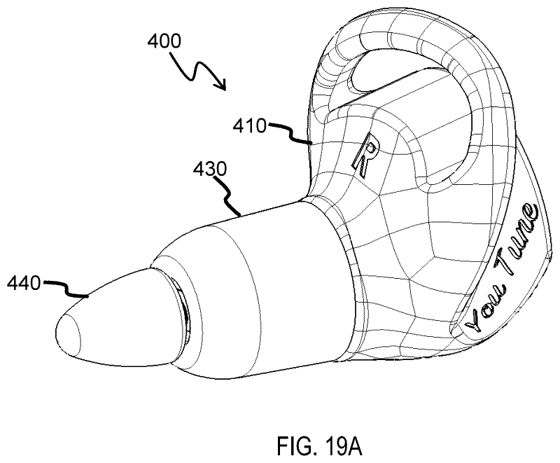

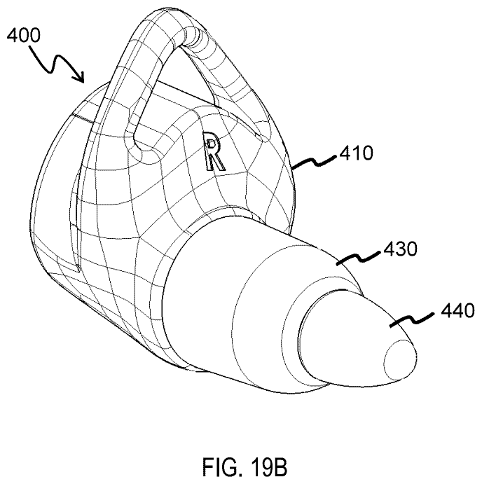

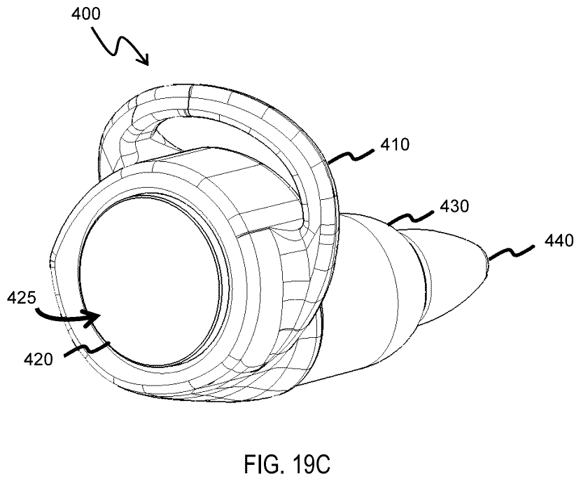

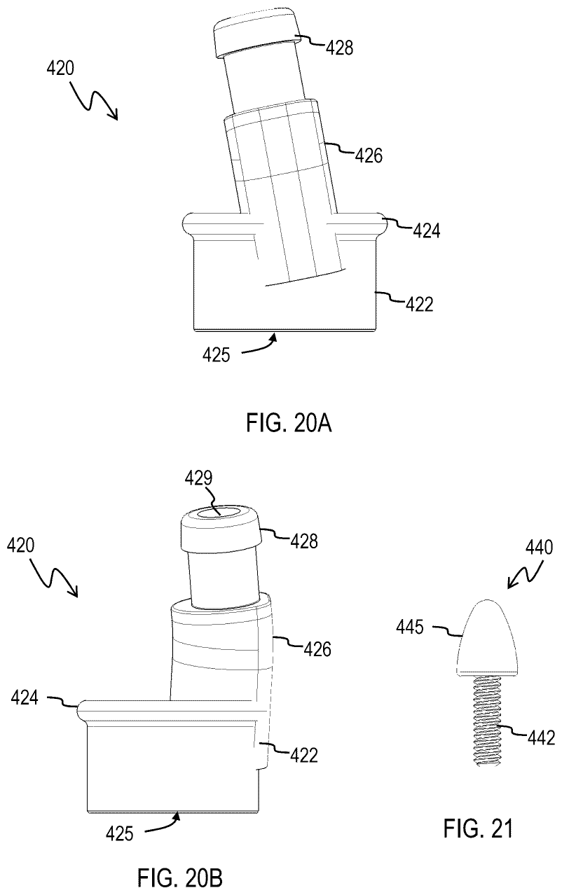

FIGS. 18A and 18B are two views of a universal-fit earpiece 400 for use with the hearing protection systems described herein. The universal-fit earpiece 400 includes a thermally conductive insert 420 surrounded by an earpiece body 410. As shown in FIGS. 19A, 19B, and 19C, thermally conductive insert 420 of the universal-fit earpiece 400 may be adapted to be covered by a comfort covering 430 such as a foam tip, for example. Referring further to FIGS. 20A, 20B, and 21, thermally conductive insert 420 includes a thermally conductive tip 440 on the end of the thermally conductive insert 420 that is inserted into the user's ear.

The thermally conductive tip 440 may be made of a thermally conductive material and may have an end portion 445 with a shape suitable for effective cooling of any user's ear, regardless of specific anatomical features of the particular person. The thermally conductive insert 420 includes an earpiece base 422 having a contact surface 425 that is essentially flat for thermal communication with the cooling module, an earpiece body 426, and a tip receiving end 428 having a threaded opening 429 for accommodating a tip threads 442 of the thermally conducive tip 440. The thermally conductive insert 420 optionally may include a seating ridge 424 adapted to hold the position of the thermally conductive insert 420 within a corresponding groove of the earpiece body 410. The thermally conductive insert 420 may further include an earpiece magnet (not shown) or, alternatively, may be made of a magnetic alloy that can be attracted by the cooling magnet of the cooling module.

The hearing protection devices according to embodiments may be powered by connecting the thermoelectric coolers to a suitable power source, such as a common battery pack. The hearing protection devices may further include a control module that adjusts the power delivered to the thermoelectric coolers and, thereby, can affect the magnitude of temperature decrease to the user's ear.

The operating principle of the hearing protection devices according to embodiments may include one or more of the following aspects. The user inserts an earpiece, specifically the thermally conductive earpiece tip, into each of the user's ears. Thereby, a thermally conductive element is disposed in the user's ear canal. The flat end of the thermally conductive earpiece tip remains exposed outside the user's ear. The user then takes hold of a headphone assembly that includes two cooling assemblies, one for each ear. The cooling assemblies are oriented such that the cooling module covers of the cooling assemblies align with the flat surfaces of the earpieces. The cooling module covers are themselves essentially flat surfaces and have magnets behind them. Accordingly, the two cooling modules magnetically lock to the earpieces. Then, the thermoelectric coolers may be activated. The thermoelectric coolers are adapted to pull heat from the ears, through the thermally conductive earpiece tips, to be rejected to the heat sinks. The fans on the cooling assemblies are activated to ensure the heat rejected to the heat sinks is further rejected to the environment. Thereby, the temperature inside the user's ear drops to therapeutic levels, such as to about 30° C. In a high-noise environment, this temperature drop may mitigate or even prevent damage to the hearing that would be expected to occur at the same level of noise, when the inside of the ear is at its normal temperature around 37° C.

Accordingly, further embodiments herein are directed to methods for mitigating hearing trauma to the ears of a user with a headphone system as previously described. The methods include inserting one earpiece into each ear of the user; magnetically attaching the two cooling assemblies of the headphone system to the thermally conductive inserts of the earpieces to establish thermal communication between the cooling modules of the cooling assemblies and the conductive tip portions of the earpieces; and activating the thermoelectric coolers of the cooling assemblies to lower the ambient temperature of the ears of the user. In embodiments, the ambient temperature of the ears of the user is lowered to approximately 30° C.

In other embodiments, the methods for mitigating hearing trauma to the ears of a user with a headphone system as previously described may include inserting the first earpiece into a left ear canal of the user; inserting the second earpiece into a right ear canal of the user; magnetically attaching the first cooling assembly to the first thermally conductive insert of the first earpiece to establish thermal communication between the first cooling module and the first conductive tip portion of the first earpiece; magnetically attaching the second cooling assembly to the second thermally conductive insert of the second earpiece to establish thermal communication between the second cooling module and the second conductive tip portion of the second earpiece; and activating the first thermoelectric cooler, the second thermoelectric cooler, or both, to decrease ambient temperature in the left ear canal, the right ear canal, or both.

In other embodiments, the methods for mitigating hearing trauma to an ear of a user with a hearing protection device includes inserting the earpiece into an ear of the user; magnetically attaching the cooling assembly to the earpiece to establish thermal communication between first cooling module and the conductive tip portion of the earpiece; and activating the thermoelectric cooler of the cooling assembly to decrease ambient temperature in the ear canal of the user.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

Unless otherwise defined, all technical and scientific terms used herein have the same meaning as commonly understood by one of ordinary skill in the art to which the invention belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A hearing protection device comprising:
a cooling assembly; and
an earpiece magnetically attachable to and detachable from the cooling assembly,
wherein:
the cooling assembly comprises a cooling module, a heat sink module in thermal communication with the cooling module, and a fan adapted to reject heat from the cooling module;
the cooling module comprises a thermoelectric cooler and a cooler magnet configured to magnetically attach to the earpiece; and
the earpiece comprises an earpiece body, a thermally conductive insert insertable into an ear canal and having a thermally conductive tip.

2. The hearing protection device of claim 1, wherein the thermally conductive insert comprises a magnetic alloy magnetically attractable to the cooler magnet.

3. The hearing protection device of claim 1, wherein the thermally conductive insert further comprises an earpiece magnet inserted into an earpiece magnet slot of the thermally conductive insert, the earpiece magnet being magnetically attractable to the cooler magnet.

4. The hearing protection device of claim 1, wherein the fan of the cooling assembly is configured to circulate air through the cooling assembly to reject the heat from the cooling module.

5. The hearing protection device of claim 1, wherein the fan of the cooling assembly is configured to aim cooling air over fins of the heat sink.

6. The hearing protection device of claim 1, wherein the thermally conductive insert comprises an earpiece base having a contact surface for thermal communication with the cooling module.

7. The hearing protection device of claim 1, wherein the thermally conductive insert further comprises a tip receiving end opposite the contact surface and a threaded opening at the tip receiving end, the threaded opening configured to accommodate tip threads of the thermally conductive tip.

8. A cooling assembly comprising:
a cooling module comprising a thermoelectric cooler and a cooler magnet adapted to magnetically attach the cooling module to a contact surface of a thermally conductive insert of an earpiece;
a cooling module cover covering the cooling module, the cooling module cover comprising a thermal transfer surface configured to establish thermal communication with the contact surface;
a heat sink module in thermal communication with the cooling module; and
a fan adapted to reject heat from the cooling module.

9. A headphone system comprising:
two cooling assemblies according to claim 8, the two cooling assemblies comprising a first cooling assembly configured to be place over a left ear of a user and a second cooling assembly configured to be placed over a right ear of the user; and
a headband connecting the two cooling assemblies.

10. The headphone system of claim 9, further comprising:
a first earpiece magnetically attachable to the first cooling assembly and comprising a first earpiece body, a first thermally conductive insert through the first earpiece body and having a first thermally conductive tip portion, and a first earpiece magnet configured to magnetically attract to the cooler magnet of the first cooling assembly; and
a second earpiece magnetically attachable to the second cooling assembly and comprising a second earpiece body, a second thermally conductive insert through the second earpiece body and having a second thermally conductive tip portion, and a second earpiece magnet configured to magnetically attract to the cooler magnet of the second cooling assembly.

11. A method for mitigating hearing trauma to an ear of a user with a hearing protection device according to claim 1, the method comprising:
inserting the earpiece into an ear of the user;
magnetically attaching the cooling assembly to the earpiece to establish thermal communication between first cooling module and the conductive tip portion of the earpiece; and
activating the thermoelectric cooler of the cooling assembly to decrease ambient temperature in the ear canal of the user.

12. A method for mitigating hearing trauma to the ears of a user with a headphone system according to claim 10, the method comprising:
inserting the first earpiece into a left ear canal of the user;
inserting the second earpiece into a right ear canal of the user;
magnetically attaching the first cooling assembly to the first thermally conductive insert of the first earpiece to establish thermal communication between the first cooling module and the first conductive tip portion of the first earpiece;
magnetically attaching the second cooling assembly to the second thermally conductive insert of the second earpiece to establish thermal communication between the second cooling module and the second conductive tip portion of the second earpiece; and
activating the first thermoelectric cooler, the second thermoelectric cooler, or both, to decrease ambient temperature in the left ear canal, the right ear canal, or both.

13. The method of claim 12, wherein the ambient temperature is decreased to approximately 30° C.
